(19) United States
(12) Patent Application Publication
(10) Pub. No.: US 2016/0046541 A1
(43) Pub. Date: Feb. 18, 2016

(54) PROCESS FOR MAKING LINEAR LONG-CHAIN ALKANES FROM RENEWABLE FEEDSTOCKS USING CATALYSTS COMPRISING HETEROPOLYACIDS

(71) Applicant: E.I. DU PONT DE NEMOURS AND COMPANY, Wilmington, DE (US)

(72) Inventors: Manish S. Kelkar, Wilmington, DE (US); Joseph E. Murphy, Woodbury, NJ (US); Joachim C. Ritter, Wilmington, DE (US); Sourav Kumar Sengupta, Wilmington, DE (US)

(21) Appl. No.: 14/771,652

(22) PCT Filed: Mar. 12, 2014
(86) PCT No.: PCT/US14/23905

§ 371 Date: Aug. 31, 2015

Related U.S. Application Data
(60) Provisional application No. 61/782,198, filed on Mar. 14, 2013.

(51) Int. Cl. C07C 1/20

(52) U.S. Cl. CPC C07C 1/20; C07C 2527/188; C07C 2523/889; C07C 2523/888; C07C 2529/076; C07C 2529/072; C07C 2523/72

(57) ABSTRACT

A hydrodeoxygenation process for producing a linear alkane from a feedstock comprising a saturated or unsaturated C₁₀₋₁₈ oxygenate that comprises an ester group, carboxylic acid group, carbonyl group and/or alcohol group is disclosed. The process comprises contacting the feedstock with a catalyst composition comprising a metal catalyst and a heteropolyacid or heteropolyacid salt, at a temperature between about 240° C. to 280° C. and a hydrogen gas pressure of at least 300 psi. The metal catalyst comprises copper in certain embodiments. By contacting the feedstock with the catalyst composition under these temperature and pressure conditions, the C₁₀₋₁₈ oxygenate is hydrodeoxygenated to a linear alkane that has the same carbon chain length as the C₁₀₋₁₈ oxygenate.

This application claims the benefit of U.S. Provisional Application Nos. 61/782,172 and 61/782,198, each filed Mar. 14, 2013, both of which are incorporated herein by reference in their entireties.

### FIELD OF THE INVENTION

This invention is in the field of chemical processing. More specifically, this invention pertains to a process for producing linear long-chain alkanes from feedstocks comprising C₁₀₋₁₈ oxygenates such as fatty acids and triglycerides.

### BACKGROUND OF THE INVENTION

Long-chain alpha,omega-dicarboxylic acids (long-chain diacids, “LCDA”), i.e., those having a carbon chain length of 9 or higher, are used as raw materials in the synthesis of a variety of chemical products and polymers (e.g., long-chain polyamides). The types of chemical processes used to make long-chain diacids have a number of limitations and disadvantages, not the least of which is the fact that these processes are based on non-renewable petrochemical feedstocks. Also, the multi-reaction conversion processes used for preparing long-chain diacids generate unwanted by-products resulting in yield losses, heavy metal wastes and nitrogen oxides which need to be destroyed in a reduction furnace.

Given the high cost and increased environmental footprint left by fossil fuels and the limited petroleum reserves in the world, there is heightened interest in using renewable sources such as fats and oils obtained from plants, animals and microbes to make chemical products and polymers such as long-chain diacids.

Long-chain diacids can be made from long-chain alkanes, which in turn can be made by converting fatty acids and triglycerides via hydrodeoxygenation (HDO). The alkane products of this reaction not only can be used to produce long-chain diacids, but are also useful as fuel by itself or in a mixture with diesel from petroleum feedstocks.

Conventional deoxygenation processes for converting renewable feedstocks to long-chain alkanes include catalytic hydrodeoxygenation, catalytic or thermal decarboxylation, catalytic decarbonylation and catalytic hydrocracking. Commercially available deoxygenation reactions are typically operated under high pressure and temperature in the presence of hydrogen gas, rendering the process expensive. A few low pressure deoxygenation processes have also been described; however, such processes suffer from several disadvantages such as low activity, poor catalyst stability, and undesirable side reactions. Typically, these processes require a high temperature and result in a high degree of decarboxylation and decarbonylation, leading to shortening of chain length of the long-chain alkane products.

For example, U.S. Pat. Appl. Publ. No. 2012-0029250 discloses a deoxygenation process that produces pentadecane (C15:0) and heptadecane (C17:0) from palmitic acid (C16:0) and oleic acid (C18:1), respectively, via decarboxylation. This process also required a reaction temperature of at least 300° C. Besides resulting in products with carbon loss, the deoxygenation process also resulted in incompletely deoxygenated products such as stearic acid, unsaturated isomers of oleic acid, and branched products. The formation of decarboxylated as well as branched products was also observed using processes disclosed in U.S. Pat. Nos. 8,193,400 and 7,999,142.

U.S. Pat. No. 8,142,527 discloses a hydrodeoxygenation process to produce diesel fuel from vegetable and animal oils requiring a reaction temperature of at least 300° C. A hydrodeoxygenation process disclosed by U.S. Pat. No. 8,026,401 required a reaction temperature of at least 400° C.

Thus, there continues to be a need for hydrodeoxygenation processes that can be carried out under conditions of low temperature, and which reliably convert the fatty acids of oils and fats from renewable resources to long-chain, linear alkanes without substantial carbon loss.

### SUMMARY OF THE INVENTION

In one embodiment, the invention concerns a hydrodeoxygenation process for producing a linear alkane from a feedstock comprising a saturated or unsaturated C₁₀₋₁₈ oxygenate that comprises a moiety selected from the group consisting of an ester group, carboxylic acid group, carbonyl group and alcohol group. This process comprises contacting the feedstock with a catalyst composition at a temperature between about 240° C. to about 280° C. and a hydrogen gas pressure of at least about 300 psi. The catalyst composition comprises (i) a metal catalyst and (ii) a heteropolyacid or heteropolyacid salt. By contacting the feedstock with the catalyst composition under these temperature and pressure conditions, the C₁₀₋₁₈ oxygenate is hydrodeoxygenated to a linear alkane that has the same carbon chain length as the C₁₀₋₁₈ oxygenate. Optionally, the hydrodeoxygenation process in this embodiment further comprises the step of recovering the linear alkane produced in the contacting step.

In a second embodiment, the C₁₀₋₁₈ oxygenate is a fatty acid or a triglyceride.

In a third embodiment, the feedstock comprises a plant oil or a fatty acid distillate thereof. The feedstock can comprise, for example, (i) a plant oil selected from the group consisting of soybean oil, palm oil and palm kernel oil; or (ii) a palm fatty acid distillate.

In a fourth embodiment, the temperature is about 260° C. The pressure is at least about 1000 psi in a fifth embodiment.

In a sixth embodiment, the metal catalyst comprises copper. This metal catalyst further comprises at least one additional metal selected from the group consisting of manganese, chromium and barium, in a seventh embodiment.

In an eighth embodiment, the heteropolyacid or heteropolyacid salt comprises tungsten. The heteropolyacid or heteropolyacid salt further comprises phosphorus or silicon in a ninth embodiment.

In a tenth embodiment, the catalyst composition comprises the heteropolyacid salt; the heteropolyacid salt is acidic and insoluble in water in this embodiment. The heteropolyacid salt in this embodiment can be a cesium-exchanged heteropolyacid, for example.

In an eleventh embodiment, the catalyst composition comprises a dry mixture of the metal catalyst and the heteropolyacid or heteropolyacid salt.

In a twelfth embodiment, the molar yield of the hydrodeoxygenation process is less than 10% for a reaction product having a carbon chain length that is one or more carbon atoms shorter than the carbon chain length of the C₁₀₋₁₈ oxygenate.

### DETAILED DESCRIPTION OF THE INVENTION

The disclosures of all patent and non-patent literature cited herein are incorporated herein by reference in their entirety.

As used herein, the term “invention” or “disclosed invention” is not meant to be limiting, but applies generally to any of the inventions defined in the claims or described herein. These terms invention, disclose invention, present invention and instant invention are used interchangeably herein.

The terms “hydrodeoxygenation” (HDO), “hydrodeoxygenation process or reaction”, “deoxygenation process or reaction” and “hydrotreating” are used interchangeably herein. Hydrodeoxygenation refers to a chemical process in which hydrogen is used to reduce the oxygen content of an oxygen-containing organic compound such as an ester, carboxylic acid, ketone, aldehyde, or alcohol. Complete hydrodeoxygenation of such compounds typically yields an alkane, in which the carbon atom(s) that previously was bonded to an oxygen atom becomes hydrogen-saturated (i.e., the carbon atom has become “hydrodeoxygenated”). For example, hydrodeoxygenation of a carboxylic acid group or an aldehyde group yields a methyl group (—CH₃), whereas hydrodeoxygenation of a ketone group yields the internal carbon moiety —CH₂—.

The hydrodeoxygenation process as described herein also reduces alkene (C═C) and alkyne (C≡C) groups to C—C groups. Thus, the hydrodeoxygenation process can also be referred to as a process of reducing sites of unsaturation in organic compounds.

As used herein, hydrodeoxygenation does not refer to a process that reduces the oxygen content of a hydrocarbon through breaking a carbon-carbon bond, such as would occur with the removal of a carboxylic acid group (i.e., decarboxylation) or carbonyl group (i.e., decarbonylation). Neither does hydrodeoxygenation herein refer to a process that incompletely reduces an oxygenated carbon moiety (e.g., reduction of a carboxylic acid group to a carbonyl or alcohol group).

The terms “alkane”, “paraffin”, and “saturated hydrocarbon” are used interchangeably herein. An alkane as used herein refers to a chemical compound that consists only of hydrogen and carbon atoms, where the carbon atoms are bonded exclusively by single bonds (i.e., they are saturated compounds). Alkanes can be linear or branched and, therefore, have methyl groups at their termini.

The terms “linear alkane”, “straight-chain alkane”, “n-alkane”, and “n-paraffin” are used interchangeably herein and refer to an alkane that has only two terminal methyl groups and for which each internal (non-terminal) carbon atom is bonded to two hydrogens and two carbons. The short-hand formula for a linear alkane is CₙH₂ₙ₊₂. Linear alkanes differ from branched alkanes, which have three or more terminal methyl groups.

The term “C₁₀₋₁₈ oxygenate” as used herein refers to a linear chain of 10-18 carbon atoms in which one or more carbon atoms is bonded to an oxygen atom (i.e., one or more oxygenated carbons). Such oxygen-bonded carbon atoms are comprised in the C₁₀₋₁₈ oxygenate in the form of one or more alcohol, carbonyl, carboxylic acid, ester, and/or ether moieties. As would be understood in the art, the carboxylic acid, ester, and/or ether moieties, if present, would be located at one or both termini of the C₁₀₋₁₈ oxygenate.

Although the C₁₀₋₁₈ oxygenate can be 10, 11, 12, 13, 14, 15, 16, 17, or 18 carbon atoms in length, it typically has an even length of 10, 12, 14, 16, or 18 carbon atoms. Examples of C₁₀₋₁₈ oxygenates as referred to herein include, but are not limited to, esters, carboxylic acids, ketones, aldehydes and alcohols.

A “saturated C₁₀₋₁₈ oxygenate” as used herein refers to a C₁₀₋₁₈ oxygenate in which the constituent carbon atoms are linked to each other by single bonds (i.e., no double or triple bonds). An example of a saturated C₁₀₋₁₈ oxygenate is stearic acid (C18:0).

An “unsaturated C₁₀₋₁₈ oxygenate” as used herein refers to a C₁₀₋₁₈ oxygenate in which one or more double (alkene) or triple (alkyne) bonds are present in the carbon atom chain of the C₁₀₋₁₈ oxygenate. Examples of unsaturated C₁₀₋₁₈ oxygenates are oleic acid (C18:1) and linoleic acid (C18:2), which contain one and two double bonds, respectively.

An “ester group” as used herein refers to an organic moiety having a carbonyl group (C═O) (defined below) adjacent to an ether linkage. The general formula of an ester group is:

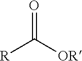

The R in the above ester formula refers to a linear chain of 9-17 carbon atoms; in this manner, the C═O carbon atom represents the tenth to eighteenth carbon atom of a C₁₀₋₁₈ oxygenate that contains an ester group. The R′ group refers to an alkyl or aryl group, for example. Examples of ester groups are found in mono-, di- and triglycerides which contain one, two, or three fatty acids, respectively, esterified to glycerol. With reference to the above formula, the R′ group of a monoglyceride would refer to the glycerol portion of the molecule. A linear alkane produced from an ester by the disclosed hydrodeoxygenation process contains the carbon atoms of the R group and the C═O group.

A “carboxylic acid group” or “organic acid group” as used herein refers to an organic moiety having a “carboxyl” or “carboxy” group (COOH). The general formula of a carboxylic acid group is:

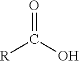

The R in the above carboxylic acid formula refers to a linear chain of 9-17 carbon atoms; in this manner, the carboxyl group (COOH) carbon atom represents the tenth to eighteenth carbon atom of a C₁₀₋₁₈ oxygenate that contains a carboxylic acid group. A linear alkane produced by the disclosed hydrodeoxygenation process retains the carboxyl group carbon atom (i.e., the product is not decarboxylated relative to the C₁₀₋₁₈ oxygenate substrate).

A “carbonyl group” as used herein refers to a carbon atom double-bonded to an oxygen atom (C═O). A carbonyl group can be located at either or both ends of the C₁₀₋₁₈ oxygenate; such a molecule could be referred to as an aldehyde. Alternatively, one or more carbonyl groups can be located within the carbon atom chain of the C₁₀₋₁₈ oxygenate; such a molecule could be referred to as a ketone.

An “alcohol group” as used herein refers to a carbon atom that is bonded to a hydroxyl (OH) group. One or more alcohol groups can be located at any carbon of the C₁₀₋₁₈ oxygenate (either or both ends, and/or one or more internal carbons of the C₁₀₋₁₈ oxygenate carbon chain).

The terms “feedstock” and “feed” are used interchangeably herein. A feedstock refers to a material comprising a saturated and/or unsaturated C₁₀₋₁₈ is oxygenate. A feedstock may be a “renewable” or “biorenewable” feedstock, which refers to a material obtained from a biological or biologically derived source.

Examples of such feedstock are materials containing monoglycerides, diglycerides, triglycerides, free fatty acids, and/or combinations thereof, and include lipids such as fats and oils. These particular types of feedstocks, which can also be referred to as “oleaginous feedstocks”, include animal fats, animal oils, poultry fats, poultry oils, plant and vegetable fats, plant and vegetable oils, yeast oils, rendered fats, rendered oils, restaurant grease, brown grease, waste industrial frying oils, fish oils, fish fats, and combinations thereof. For feedstocks comprising fat or oil, it would be understood by one of skill in the art that all or most of the C₁₀₋₁₈ oxygenate is comprised in the feedstock in the form of an ester (fatty acid esterified to glycerol). Hydrodeoxygenation of such C₁₀₋₁₈ oxygenates according to the disclosed process involves the complete reduction of the ester group of the esterified fatty acid, which in part entails breaking the ester linkage between the fatty acid and the glycerol molecule.

Alternatively, a feedstock can refer to a petroleum- or fossil fuel-derived material comprising a saturated or unsaturated C₁₀₋₁₈ oxygenate.

The terms “fatty acid distillate” and “fatty acid distillate of an oil” as used herein refer to a composition comprising the fatty acids of a particular type of oil. For example, a palm fatty acid distillate comprises fatty acids that are present in palm oil. Fatty acid distillates commonly are byproducts of plant oil refining processes.

The terms “moiety”, “chemical moiety”, “functional moiety”, and “functional group” are used interchangeably herein. A moiety as used herein refers to a carbon group comprising a carbon atom bonded to an oxygen atom. Examples of a moiety as used herein include ester, carboxylic acid, carbonyl and alcohol groups.

The terms “percent by weight”, “weight percentage (wt %)” and “weight-weight percentage (% w/w)” are used interchangeably herein. Percent by weight refers to the percentage of a material on a mass basis as it is comprised in a composition or mixture. For example, percent by weight refers to the percentage of a metal by mass that is present in a catalyst as described herein. Except as otherwise noted, all the percentage amounts of metals or other materials disclosed herein refer to percent by weight of the metals or other materials in catalysts.

The term “catalyst composition” as used herein refers to a composition comprising a metal catalyst component and a heteropolyacid or heteropolyacid salt component. The metal catalyst component may comprise one or more active metals such as copper; the one or more active metals may be supported (i.e., the metal catalyst component may be a supported metal catalyst). The catalyst composition can increase the rate of C₁₀₋₁₈ oxygenate hydrodeoxygenation without itself being consumed or undergoing a chemical change.

The terms “solid support”, “support”, and “catalyst support” are used interchangeably herein. A solid support refers to the material to which an active catalyst component (e.g., a metal and/or a heteropolyacid) is anchored or bound. Catalysts described herein that contain a solid support are examples of “supported catalysts”. The metal catalyst component of the catalyst composition may itself be a supported metal catalyst. In turn, the heteropolyacid or heteropolyacid salt component in certain embodiments of the catalyst composition may be supported on the metal catalyst or supported metal catalyst.

A “heteropolyacid” as used herein refers to a compound having a center element and peripheral elements to which oxygen is bonded (e.g., H₃PW₁₂O₄₀, where P is the central element and W is the peripheral element). Some heteropolyacid structures have been described; e.g., Keggin, Wells-Dawson and Anderson-Evans-Perloff structures. The center element in certain embodiments can be Si, P, Ge, As, B, Ti, Ce, Co, Ni, Al, Ga, Bi, Cr, Sn, or Zr. Examples of the peripheral element can be metals such as W, Mo, V, or Nb. A heteropolyacid is soluble in water. A “heteropolyacid salt” as used herein is a heteropolyacid that is ionically linked to a cation (e.g., cesium cation). A heteropolyacid salt can also be referred to as a “cation-exchanged heteropolyacid”. The heteropolyacid salts referred to herein are insoluble in water.

The terms “specific surface area”, “surface area”, and “solid support surface area” are used interchangeably herein. The specific surface area of a solid support is expressed herein as square meters per gram of solid support (m²/g). The specific surface area of the solid supports disclosed herein can be measured, for example, using the Brunauer, Emmett and Teller (BET) method (Brunauer et al., J. Am. Chem. Soc. 60:309-319; incorporated herein by reference).

The terms “impregnation” and “loading” are used interchangeably herein. Impregnation refers to the process of rendering a metal salt or other compound (e.g., heteropolyacid) into a finely divided form or layer on a solid support. This process in certain embodiments involves drying down a mixture containing a solid support and a solution of a metal salt or other water-dissolvable compound. The dried product can be referred to as a “pre-catalyst”.

The terms “calcining” and “calcination” as used herein refer to a thermal treatment of a pre-catalyst. This process can convert a dried metal salt of a pre-catalyst to a metallic or oxide state, for example. The thermal treatment can be performed in either an inert or active atmosphere.

The terms “molar yield”, “reaction yield”, and “yield” are used interchangeably herein. Molar yield refers to the amount of a product obtained in a chemical reaction as measured on a molar basis. This amount can be expressed as a percentage; i.e., the percent amount of a particular product in all of the reaction products.

The terms “reaction mix”, “reaction mixture”, and “reaction composition” are used interchangeably herein. A reaction mix can minimally comprise a feedstock (substrate), metal catalyst, heteropolyacid or heteropolyacid salt, and a solvent. A reaction mix can describe the mix as it exists before or during application of the temperature and pressure hydrodeoxygenation conditions.

Disclosed herein is a hydrodeoxygenation process that can be carried out under low temperature conditions, and which converts C₁₀₋₁₈ oxygenates in feedstocks to linear alkanes without substantial carbon loss. Therefore, the process produces fewer undesirable by-products and is more economical since it can be run at a low temperature.

Embodiments of the disclosed invention concern a hydrodeoxygenation process for producing a linear alkane from a feedstock comprising a saturated or unsaturated C₁₀₋₁₈ oxygenate that comprises a moiety selected from the group consisting of an ester group, carboxylic acid group, carbonyl group and alcohol group. This process comprises contacting the feedstock with a catalyst composition at a temperature between about 240° C. to about 280° C. and a hydrogen gas pressure of at least about 300 psi. The catalyst composition comprises (i) a metal catalyst and (ii) a heteropolyacid or heteropolyacid salt. By contacting the feedstock with the catalyst composition under these temperature and pressure conditions, the C₁₀₋₁₈ oxygenate is hydrodeoxygenated to a linear alkane that has the same carbon chain length as the C₁₀₋₁₈ oxygenate. Optionally, the hydrodeoxygenation process further comprises the step of recovering the linear alkane produced in the contacting step.

The feedstock used in certain embodiments of the disclosed invention may comprise a material comprising one or more monoglycerides, diglycerides, triglycerides, free fatty acids, and/or combinations thereof, and include lipids such as fats and oils. Examples of such feedstocks include fats and/or oil derived from animals, poultry, fish, plants, microbes, yeast, fungi, bacteria, algae, euglenoids and stramenopiles. Examples of plant oils include canola oil, corn oil, palm kernel oil, cheru seed oil, wild apricot seed oil, sesame oil, sorghum oil, soy oil, rapeseed oil, soybean oil, colza oil, tall oil, sunflower oil, hempseed oil, olive oil, linseed oil, coconut oil, castor oil, peanut oil, palm oil, mustard oil, cottonseed oil, camelina oil, jatropha oil and crambe oil. Other feedstocks include, for example, rendered fats and oil, restaurant grease, yellow and brown greases, waste industrial frying oil, tallow, lard, train oil, fats in milk, fish oil, algal oil, yeast oil, microbial oil, yeast biomass, microbial biomass, sewage sludge and soap stock.

Derivatives of oils such as fatty acid distillates are other examples of feedstocks that can be used in certain embodiments of the invention. Plant oil distillates (e.g., palm fatty acid distillate) are preferred examples of fatty acid distillates. A fatty acid distillate of any of the fats and oils disclosed herein may be used in certain embodiments of the invention.

The feedstock comprises a plant oil or a fatty acid distillate thereof in a preferred embodiment of the invention. In another preferred embodiment, the feedstock comprises (i) a plant oil selected from the group consisting of soybean oil, palm oil and palm kernel oil; or (ii) a palm fatty acid distillate (e.g., produced from refining crude palm oil).

Palm oil is derived from the mesocarp (pulp) of the fruit of the oil palm, whereas palm kernel oil is derived from the kernel of the oil palm. The fatty acids comprised in palm oil typically include palmitic acid (˜44%), oleic acid (˜37%), linoleic acid (˜9%), stearic acid and myristic acid. The fatty acids comprised in palm kernel oil typically include lauric acid (˜48%), myristic acid (˜16%), palmitic acid (˜8%), oleic acid (˜15%), capric acid, caprylic acid, stearic acid and linoleic acid. Soybean oil typically comprises linoleic acid (˜55%), palmitic acid (˜11%), oleic acid (˜23%), linolenic acid and stearic acid.

Fossil fuel-derived and other types of feedstocks that can be used in certain embodiments of the disclosed invention include petroleum-based products, spent motor oils and industrial lubricants, used paraffin waxes, coal-derived liquids, liquids derived from depolymerization of plastics such as polypropylene, high density polyethylene, and low density polyethylene; and other synthetic oils generated as byproducts from petrochemical and chemical processes.

Examples of other feedstocks are described in U.S. Pat. Appl. Publ. No. 2011-0300594, which is incorporated herein by reference.

The C₁₀₋₁₈ oxygenate comprised in the feedstock may be a fatty acid or a triglyceride. The feedstock may comprise one or more fatty acids that are in the free form (i.e., non-esterified) or that are esterified. Esterified fatty acids may be those comprised within a glyceride molecule (i.e., in a fat or oil) or fatty acid alkyl ester (e.g., fatty acid methyl ester or fatty acid ethyl ester), for example. The fatty acid(s) may be saturated or unsaturated. Examples of unsaturated fatty acids are monounsaturated fatty acids (MUFA) if only one double bond is present in the fatty acid carbon chain, and polyunsaturated fatty acids (PUFA) if the fatty acid carbon chain has two or more double bonds. The carbon chain length of a fatty acid C₁₀₋₁₈ oxygenate in the feedstock may be 10, 11, 12, 13, 14, 15, 16, 17, or 18 carbon atoms. Preferably, the carbon chain length is 10, 12, 14, 16, or 18 carbon atoms. Another preferred fatty acid length is 16-18 carbon atoms. Examples of fatty acids that can be in the feedstock are provided in Table 1.

TABLE 1
Examples of Saturated and Unsaturated Fatty Acids
that May Be Comprised in Feedstocks

| | | Shorthand |
|---|---|---|
| Common Name | Chemical Name | Notation |
| Capric | decanoic | 10:0 |
| Undecylic | undecanoic | 11:0 |
| Lauric | dodecanoic | 12:0 |
| Tridecylic | tridecanoic | 13:0 |
| Myristic | tetradecanoic | 14:0 |
| Myristoleic | tetradecenoic | 14:1 |
| Pentadecylic | pentadecanoic | 15:0 |
| Palmitic | hexadecanoic | 16:0 |
| Palmitoleic | 9-hexadecenoic | 16:1 |
| | hexadecadienoic | 16:2 |
| Margaric | heptadecanoic | 17:0 |
| Stearic | octadecanoic | 18:0 |
| Oleic | cis-9-octadecenoic | 18:1 |
| Linoleic | cis-9,12-octadecadienoic | 18:2 omega-6 |
| gamma-linolenic | cis-6,9,12- | 18:3 omega-6 |
| | octadecatrienoic | |
| alpha-linolenic | cis-9,12,15- | 18:3 omega-3 |
| | octadecatrienoic | |
| Stearidonic | cis-6,9,12,15- | 18:4 omega-3 |
| | octadecatetraenoic | |

Although the oxygenates comprised in the feedstocks used in the disclosed invention have a length of 10-18 carbon atoms, other oxygenates with a carbon length outside this range may also be present in the feedstock. For example, the glycerides and free fatty acids of the fats and oils that can be used as feedstock may also contain carbon chains of about 8 to 24 carbon atoms in length. In other words, the feedstock need not comprise only C₁₀₋₁₈ oxygenates.

The C₁₀₋₁₈ oxygenates represented by lipids and free fatty acids comprise ester and carboxylic acid moieties, respectively. Other types of C₁₀₋₁₈ oxygenates may be comprised in the feedstock such as those C₁₀₋₁₈ oxygenates containing one or more carbonyl and/or alcohol moieties. Still other types of C₁₀₋₁₈ oxygenates may contain two or more of any of the above moieties. Examples include C₁₀₋₁₈ oxygenates comprising two or more alcohol moieties (e.g., diols), carbonyl moieties (e.g., diketones or dialdehydes), carboxylic acid moieties (dicarboxylic acids), or ester moieties (diesters). C₁₀₋₁₈ oxygenates comprising alcohol and carbonyl moieties (e.g., hydroxyketones and hydroxyaldehydes), alcohol and carboxylic acid moieties (e.g., hydroxycarboxylic acids), alcohol and ester moieties (e.g., hydroxyesters), carbonyl and carboxylic acid moieties (e.g., keto acids), or carbonyl and ester moieties (e.g., keto esters) are other example components of feedstocks that can be used in embodiments of the disclosed invention. The C₁₀₋₁₈ oxygenate in certain embodiments does not comprise any aromatic groups (e.g., phenol).

The feedstock may contain one or more C₁₀₋₁₈ oxygenates linked together by two or more ester and/or ether linkages. Such C₁₀₋₁₈ oxygenates are unlinked from each other during the disclosed hydrodeoxygenation process; the removal of oxygen from such molecules destroys the ester and/or ether linkages. Similarly, the fatty acid C₁₀₋₁₈ oxygenates as contained in a glyceride feedstock are unlinked from the glycerol component of the glyceride during the disclosed hydrodeoxygenation process since the fatty acid ester linkages are destroyed by the removal of oxygen. Therefore, different types of linear alkanes can be produced from feedstocks containing two or more different C₁₀₋₁₈ oxygenates, even if the C₁₀₋₁₈ oxygenates are linked by ester and/or ether linkages. All these types of C₁₀₋₁₈ oxygenates may be constituent components of the feedstock.

The linear chain of the C₁₀₋₁₈ oxygenate is not linked to any alkyl or aryl branches via a carbon-carbon bond from one of the carbon atoms of the linear chain. For example, while palmitic acid is a C₁₀₋₁₈ oxygenate in certain embodiments, palmitic acid having an alkyl group substitution (e.g., 15-methyl palmitic acid) at one of its —CH₂— moieties is not a type of C₁₀₋₁₈ oxygenate as described herein. The disclosed hydrodeoxygenation process does not involve isomerization events that involve removing and/or adding carbon-carbon bonds to a carbon of the C₁₀₋₁₈ oxygenate. Therefore, branched alkane products such as isodecanes, isododecanes, isotetradecanes, isohexadecanes and isooctadecanes are not produced.

The C₁₀₋₁₈ oxygenate may constitute the feedstock itself in certain embodiments of the disclosed invention. An example of such a feedstock is a pure or substantially pure preparation of a particular fatty acid. Alternatively, the feedstock may comprise multiple separate C₁₀₋₁₈ oxygenates (i.e., distinct molecules that are not linked to each other). Mixtures of any of the above feedstocks may be used as co-feed components in the disclosed hydrodeoxygenation process.

A linear alkane is produced from a saturated or unsaturated C₁₀₋₁₈ oxygenate in the disclosed hydrodeoxygenation process. The linear alkanes produced in certain embodiments include decane, undecane, dodecane, tridecane, tetradecane, pentadecane, hexadecane, heptadecane and octadecane, where those of these linear alkanes having an even carbon atom number are produced in preferred embodiments.

As discussed above, hexadecane is a linear alkane produced in certain embodiments of the disclosed hydrodeoxygenation process. Various C₁₆ oxygenates can be used as feedstock to produce hexadecane, including hexadecanol (e.g., cetyl alcohol), hexadecyl aldehyde, hexadecyl ketone, palmitic acid, palmityl palmitate, and/or any other C₁₆ oxygenate in which one or more carbon atoms of the C16 chain is bonded to an oxygen atom, for example. The feedstock in certain embodiments may comprise any of these various C₁₆ oxygenates. For example, the feedstock may be an oil or fat comprising palmitic acid (i.e., contain a palmitoyl group) or palmitoleic acid (i.e., contain a 9-hexadecenoyl group).

Octadecane is a linear alkane produced in certain embodiments of the disclosed hydrodeoxygenation process. Various C₁₈ oxygenates can be used as feedstock to produce octadecane, including octadecanol (e.g., stearyl alcohol), octadecyl aldehyde, octadecyl ketone, stearic acid, stearyl stearate, and/or any other C₁₈ oxygenate in which one or more carbon atoms of the C18 chain is bonded to an oxygen atom, for example. The feedstock in certain embodiments may comprise any of these various C₁₈ oxygenates. For example, the feedstock may be an oil or fat comprising stearic acid (i.e., contain a stearoyl group), oleic acid (i.e., contain a 9-octadecenoyl group), or linoleic acid (i.e., contain a 9,12-octadecadienoyl group).

The molar yield of the linear alkane in certain embodiments of the disclosed invention is at least about 10%, 11%, 12%, 13%, 14%, 15%, 16%, 17%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The carbon chain length of the linear alkane product of the disclosed hydrodeoxygenation process is the same carbon chain length of the C₁₀₋₁₈ oxygenate. For example, where the C₁₀₋₁₈ oxygenate is palmitic acid, the resulting linear alkane is hexadecane; both palmitic acid and hexadecane have a carbon chain length of sixteen carbon atoms. The linear alkane produced in the disclosed process therefore represents the completely hydrogen-saturated, reduced form of the C₁₀₋₈ oxygenate in the feedstock. For example, the disclosed hydrodeoxygenation process produces decane from capric acid; dodecane from lauric acid; tetradecane from myristic acid and myristoleic acid; hexadecane from palmitic acid and palmitoleic acid; and octadecane from stearic acid, oleic acid and linoleic acid. These linear alkanes are produced whether the fatty acids are free or esterified. A C₁₀₋₁₈ oxygenate that is linked to one or more other components via ester and/or ether linkages yields a linear alkane during the disclosed process that represents the completely hydrogen-saturated, reduced form of the C₁₀₋₁₈ oxygenate.

In certain embodiments of the disclosed invention, the molar yield is less than about 10% for a reaction product having a carbon chain length that is one or more carbon atoms shorter than the carbon chain length of the C₁₀₋₁₈ oxygenate. In other embodiments, the molar yield is less than about 1%, 2%, 3%, 4%, 5%, 6%, 7%, 8%, or 9% for a reaction product having a carbon chain length that is one or more carbon atoms shorter than the carbon chain length of the C₁₀₋₁₈ oxygenate. The low level of such byproducts using the disclosed invention reflects a low level of carbon loss from the C₁₀₋₁₈ oxygenate by decarboxylation and/or decarbonylation events during the hydrodeoxygenation reaction. Therefore, the disclosed process does not significantly break carbon-carbon bonds of the C₁₀₋₁₈ oxygenate.

The molar yield of other types of byproducts in certain embodiments of the disclosed invention is less than about 1%, 2%, 3%, 4%, 5%, 6%, 7%, 8%, 9%, 10%, 11%, 12%, 13%, 14%, or 15%. Such other byproducts include products that represent incompletely reduced forms of the C₁₀₋₁₈ oxygenate that retain one or more oxygenated carbon atoms (e.g., alcohol group, carbonyl group, carboxylic acid group, ester group, ether group), and/or one or more points of unsaturation.

The disclosed hydrodeoxygenation process in certain embodiments can be tested with respect to its ability to convert dodecanol into dodecane. In other words, a hydrodeoxygenation process for converting C₁₆ or C₁₈ oxygenates to alkanes can be tested using lauric acid or dodecanol as the feedstock; such processes when tested on lauric acid or dodecanol can have molar yields of dodecane as listed above for linear alkanes. Similarly, such processes when tested on lauric acid or dodecanol can have molar yields of byproducts less than about 1%, 2%, 3%, 4%, 5%, 6%, 7%, 8%, 9%, 10%, 11%, 12%, 13%, 14%, or 15%.

The linear alkanes produced in the disclosed process can be isolated by close-cut distillation, for example. If necessary, selective adsorption with molecular sieves can be used to further purify the linear alkanes from those reaction byproducts that are bulkier than the linear alkanes. Molecular sieves can comprise synthetic zeolites having a series of central cavities interconnected by pores. The pores have diameters large enough to permit passage of linear alkanes, but not large enough to allow passage of branched byproducts. Commercial isolation processes using molecular sieves include IsoSiv™ (Dow Chemical Company), Molex™ (UOP LLC) and Ensorb™ (Exxon Mobil Corporation), for example.

The instant invention includes the step of contacting the feedstock comprising a C₁₀₋₁₈ oxygenate with a catalyst composition at a temperature between about 240° C. to about 280° C. and a hydrogen gas pressure of at least about 300 psi.

The step of contacting the feedstock with the catalyst may be performed in a reaction vessel or any other enclosure known in the art that allows performing a reaction under controlled temperature and pressure conditions. For example, the contacting step is performed in a packed bed reactor, such as a plug flow, tubular or other fixed bed reactor. It should be understood that the packed bed reactor may be a single packed bed or comprise multiple beds in series and/or in parallel. Alternatively, the contacting step can be performed in a slurry reactor, including batch reactors, continuously stirred tank reactors, and/or bubble column reactors. In slurry reactors, the catalyst may be removed from the reaction mixture by filtration or centrifugal action. The size/volume of the reaction vessel should be suitable for handling the chosen amount of feedstock and catalyst.

The contacting step may be performed in any continuous or batch processing system as known in the art. A continuous process may be multi-stage using a series of two or more reactors in series. Fresh hydrogen may be added at the inlet of each reactor in this type of system. A recycle stream may also be used to help maintain the desired temperature in each reactor. The reactor temperature may also be controlled by controlling the fresh feedstock temperature and the recycle rate.

In certain embodiments, the contacting step may comprise agitating or mixing the feedstock and catalyst before and/or while the reaction components are subjected to the above temperature and hydrogen gas pressure conditions. Agitation can be performed using a mechanical stirrer, or in a slurry reactor system, for example.

The contacting step in certain embodiments may be performed in a solvent, such as an organic solvent or water. The solvent may consist of one type of solvent that is pure or substantially pure (e.g., >99% or >99.9% pure) or comprise two or more different solvents mixed together. The solvent may be homogeneous (e.g., single-phase) or heterogeneous (e.g., two or more phases). In a preferred embodiment, the feedstock and the catalyst are contacted in an organic solvent; thus certain embodiments exclude water and other aqueous solvents. The organic solvent used in certain embodiments may be non-polar or polar. The organic solvent comprises tetradecane, hexadecane, or dodecane in another embodiment. Alternatively, the organic solvent may be another alkane such as one having a chain length of 6 to 18 carbon atoms. The organic solvent may be selected on the basis of its ability to dissolve hydrogen. For example, the solvent can have a relatively high solubility for hydrogen so that substantially all the hydrogen provided by the hydrogen gas pressure is in solution before and/or during the disclosed hydrodeoxygenation process. The heteropolyacid or heteropolyacid salt is not soluble in an organic solvent in certain embodiments.

Certain embodiments of the invention use a solvent to substrate ratio of at least about 2:1, 3:1, 4:1, 5:1, 6:1, 7:1, or 8:1. This ratio can be determined on a weight-weight basis. A 4:1 solvent to substrate ratio could be prepared using 4 kg of tetradecane to every 1 kg of the C₁₀₋₁₈ oxygenate, for example.

The contacting step of the disclosed process is performed at a temperature between about 240° C. to about 280° C. and a hydrogen gas pressure of at least about 300 psi. The temperature in certain embodiments may be about 200° C., 210° C., 220° C., 230° C., 240° C., 245° C., 250° C., 255° C., 260° C., 265° C., 270° C., 275° C., or 280° C. Alternatively, the temperature is between about 200° C. to about 280° C., between about 200° C. to about 260° C., between about 220° C. to about 260° C., or between about 240° C. to about 260° C. The temperature is about 260° C. in other embodiments of the disclosed invention. The hydrogen gas pressure in certain embodiments may be at least about 300 psi, 400 psi, 500 psi, 600 psi, 700 psi, 800 psi, 900 psi, 1000 psi, 1100 psi, or 1200 psi. Alternatively, the hydrogen gas pressure in certain embodiments is between about 300 psi to about 1000 psi.

In certain embodiments of the disclosed invention, the feedstock and catalyst composition are contacted in the above temperature and hydrogen pressure conditions for about 2, 3, 4, 5, 6, 7, 8, 9, 10, 11, 12, 13, 14, 15, 16, 17, 18, 19 or 20 hours. The feedstock and catalyst composition can be subjected to the temperature and hydrogen pressure conditions for a continuous period of time, for example.

In certain embodiments, the feedstock is contacted with hydrogen to form a feedstock/hydrogen mixture in advance of contacting the feedstock with the catalyst composition. In other embodiments, a solvent or diluent is added to the feedstock in advance of contacting the feedstock with hydrogen and/or catalyst composition. For example, after forming a feedstock/solvent mixture, it may then be contacted with hydrogen to form a feedstock/solvent/hydrogen mixture which is then contacted with the catalyst composition.

A wide range of suitable catalyst concentrations may be used in the disclosed process, where the amount of catalyst per reactor is generally dependent on the reactor type. For a fixed bed reactor, the volume of catalyst per reactor will be high, while in a slurry reactor, the volume will be lower. Typically, in a slurry reactor, the catalyst will make up 0.1 to about 30 wt % of the reactor contents.

The present invention includes the step of contacting the feedstock comprising a C₁₀₋₁₈ oxygenate with a catalyst composition comprising (i) a metal catalyst and (ii) a heteropolyacid or heteropolyacid salt. The metal catalyst comprises copper (Cu) in certain embodiments of the disclosed hydrodeoxygenation process. The weight percentage of copper or form thereof (e.g., copper oxide) comprised in the metal catalysts can be at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%, for example. Alternatively, there may be about 40-60% (e.g., about 47-56%) copper by weight in the metal catalyst. The copper may be elemental copper, a copper oxide, or a copper salt in certain embodiments. The copper in a copper oxide and/or copper salt component of the metal catalyst may be in the cuprous (Copper I) or cupric (Copper II) oxidation state. For example, CuO (i.e., cupric oxide) can be the copper component.

The metal catalyst in certain embodiments of the invention may contain other metals. Such other metals may be included in addition to copper. For example, the metal catalyst may further comprise at least one additional metal selected from the group consisting of manganese (Mn), chromium (Cr) and barium (Ba). One of these metals, or any combination thereof, may be used. The metal catalyst can comprise any two, any three or all four of Cu, Ba, Mn and Cr. For example, the metal catalyst may comprise (i) Cu and Mn; (ii) Cu, Ba and Cr; or (iii) Cu, Ba, Mn and Cr. Certain embodiments of the metal catalyst contain at most two, three, or four of these metal components (i.e., no other components, except a support material if provided). In alternative embodiments, the metal catalyst comprises only copper as the metal component (i.e., no other components, except a support material if provided).

The weight percentage of an additional metal such as Mn, Cr and/or Ba (or form thereof) in the metal catalyst component of the catalyst composition can be at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, for example. The metal catalyst may contain, for example, about 4-10% Mn, 2-6% Ba, and/or 34%-46% Cr. Examples of metal catalysts may contain (i) about 56% Cu and about 10% Mn; (ii) about 47% Cu, about 6% Ba and about 34% Cr; or (iii) about 47% Cu, about 2% Ba, about 4% Mn and about 46% Cr.

The additional metal may be in an elemental form, oxide form, or salt form, for example. The manganese in an Mn oxide and/or Mn salt component of the metal catalyst may be in the Manganese II, II/III, IV, or VII oxidation state. For example, MnO₂ (i.e., Manganese-IV oxide) can be the Mn component. The chromium in a Cr oxide and/or Cr salt component of the metal catalyst may be in the Chromium II, III, IV, or VI oxidation state. For example, Cr₂O₃ (i.e., Chromium-Ill oxide) can be the Cr component. The barium in a Ba oxide and/or Ba salt component of the metal catalyst may be in the divalent state (Ba²⁺). For example, BaO (i.e., barium oxide) can be the Ba component. The metal catalyst may contain (i) CuO and MnO₂; (ii) CuO, BaO and Cr₂O₃, or (iii) CuO, BaO, Cr₂O₃ and MnO₂, for example. Also for example, the metal catalyst may contain (i) about 56% CuO and about 10% MnO₂; (ii) about 47% CuO, about 6% BaO and about 34% Cr₂O₃, or (iii) about 47% CuO, about 2% BaO, about 46% Cr₂O₃ and about 4% MnO₂.

The metal catalysts as described herein can be prepared via co-precipitation techniques, for example. Examples of metal catalysts that can be used in certain embodiments include CuO/MnO₂/Al₂O₃, BaO/CuO/Cr₂O₃/SiO₂, BaO/CuO/MnO₂/Cr₂O₃ and CuO/SiO₂.

Also, the metal catalysts as described herein can be prepared using any of a variety of ways known in the art (e.g., Pinna, 1998, Catalysis Today 41:129-137; Catalyst Preparation: Science and Engineering, Ed. John Regalbuto, Boca Raton, Fla.: CRC Press, 2006; Mul and Moulijn, Chapter 1: Preparation of supported metal catalysts, In: Supported Metals in Catalysis, 2nd Edition, Eds. J. A. Anderson and M. F. Garcia, London, UK: Imperial College Press, 2011; Acres et al., The design and preparation of supported catalysts, In: Catalysis: A Specialist Periodical Report, Eds. D. A. Dowden and C. C. Kembell, London, UK: The Royal Society of Chemistry, 1981, vol. 4, pp. 1-30). It is desirable that the catalyst composition prepared by a chosen method be active, selective, recyclable, and mechanically and thermochemically stable during the disclosed hydrodeoxygenation process.

The metal catalyst in the catalyst composition of the disclosed invention can comprise a support in certain embodiments (i.e., supported metal catalyst). Various solid supports as known in the art can be comprised as part of the metal catalyst, including one or more of WO₃, Al₂O₃ (alumina), TiO₂ (titania), TiO₂—Al₂O₃, ZrO₂, tungstated ZrO₂, SiO₂, SiO₂—Al₂O₃, SiO₂—TiO₂, V₂O₅, MoO₃, or carbon, for example. In a preferred embodiment, the solid support comprises Al₂O₃ or SiO₂. The solid support may therefore comprise an inorganic oxide, metal oxide or carbon. Other examples of solid supports that may be used include day (e.g., montmorillonite) and zeolite (e.g., H—Y zeolite). The support material used in the catalyst such as those described above may be basic (≧pH 9.5), neutral, weakly acidic (pH between 4.5 and 7.0), or acidic (≦pH 4.5). Additional examples of solid supports that can be used in certain embodiments of the disclosed invention are described in U.S. Pat. No. 7,749,373 which is incorporated herein by reference.

The solid support used in certain embodiments of the disclosed invention may be porous, thereby increasing the surface area onto which the metal catalyst is attached. For example, the solid support can comprise pores and have (i) a specific surface area that is at least 10 m²/g and optionally less than or equal to 280 m²/g, wherein the pores have a diameter greater than 500 angstroms and the pore volume of the support is at least 10 ml/100 g; or (ii) a specific surface area that is at least 50 m²/g and optionally less than or equal to 280 m²/g, wherein the pores have a diameter greater than 70 angstroms and the pore volume of the support is at least 30 ml/100 g. The specific surface area of the support in a supported metal catalyst component of the catalyst composition can be, for example, about or at least about 10, 20, 30, 40, 50, 60, 70, 80, 90, 100, 110, 120, 130, 140, 150, 160, 170, 180, 190, 200, 210, 220, 230, 240, 250, 260, 270, or 280 m²/g. Preparing a porous solid support with a particular specific surface area can be performed by modulating pore diameter and volume as known in the art (e.g., Trimm and Stanislaus, Applied Catalysis 21:215-238; Kim et al., Mater. Res. Bull. 39:2103-2112; Grant and Jaroniec, J. Mater. Chem. 22:86-92).

Solid supports for preparing the catalysts used in certain embodiments of the disclosed invention are available from a number of commercial sources, including Süd-Chemie (Louisville, Ky.), Johnson Matthey, Inc. (West Deptford, N.J.), BASF (Iselin, N.J.), Evonik (Calvert City, Ky.) and Sigma-Aldrich (St. Louis, Mo.), for example.

Supported metal catalysts in certain embodiments can be in the form of particles such as shaped particles. Catalyst particles can be shaped as cylinders, pellets, spheres, or any other shape. Cylinder-shaped catalysts may have hollow interiors, with or without one or more reinforcing ribs. Other particle shapes that may be used include trilobe, cloverleaf, cross, “C”-shaped, rectangular- and triangular-shaped tubes, for example. Alternatively, supported metal catalyst may be in the form of powder or larger sized cylinders or tablets. The size (diameter×height) of cylinders can be about 2×2 mm, 2.5×2.5 mm, 3×3 mm, 3.5×3.5 mm, 4×4 mm, 4.5×4.5 mm, or 5×5 mm, for example.

Any means known in the art for supporting a metal catalyst can be used, if a supported metal catalyst is used in the catalyst composition. For example, the metal catalyst used in certain embodiments may be prepared through sequential impregnation of a solid support with the selected metals. Alternatively, each of the selected metals can be impregnated onto the solid support at the same time, without sequential impregnation. In certain embodiments, a metal can be impregnated onto a supported metal catalyst obtained from a commercial source.

The impregnation of each metal onto the solid support in preparing certain catalysts for use in the disclosed process may be performed by mixing the solid support with a metal salt solution, drying this mixture at a suitable temperature (e.g., 100 to 120° C.) for a suitable amount of time to obtain a dried product, and then calcining the dried product at a suitable temperature (e.g., 300 to 400° C.) for a suitable amount of time. The supported metal catalyst prepared by this procedure may then be impregnated with another metal. Alternatively, the impregnation of each metal onto the solid support may be performed by mixing the solid support with a metal salt solution, drying this mixture as above, mixing the dried product with another metal salt solution, drying this mixture as above, and then calcining the dried product as above. Either of the above procedures can be adapted accordingly to load additional metals onto the solid support.

Metal-comprising salts known in the art to be useful in preparing supported metal catalysts can be used to prepare the catalyst following an impregnation-calcining procedure. Examples of such useful salts include nitrates, halides (e.g., chloride, bromide), acetates and carbonates.

The disclosed invention includes the step of contacting the feedstock comprising a C₁₀₋₁₈ oxygenate with a catalyst composition comprising (i) a metal catalyst and (ii) a heteropolyacid or heteropolyacid salt. The heteropolyacid as known in the art is a compound having a center element and peripheral elements to which oxygen is bonded. The center element can be Si, P, Ge, As, B, Ti, Ce, Co, Ni, Al, Ga, Bi, Cr, Sn, or Zr, for example. Examples of the peripheral element can be metals such as W, Mo, V, or Nb.

Some heteropolyacid structures have been described; e.g., Keggin, Wells-Dawson and Anderson-Evans-Perloff structures.

The heteropolyacid in certain embodiments can have a chemical structure represented by one of the following formulae:

HₖXM₁₂O₄₀ or (I)

AⱼHₖXM₁₂O₄₀, wherein (II)

X (center element) is Si, P, Ge, As, B, Ti, Ce, Co, Ni, Al, Ga, Bi, Cr, Sn, or Zr;
M (peripheral element) is independently Mo, W, V, or Nb;
A (see formula II) is an alkali metal element, alkaline earth metal element, organic amine cation, or a combination thereof;
k is 0-4;
j is 0-4; and
at least one of j and k is greater than 0.

It would be understood in the art that formula II represents a heteropolyacid salt or a cation-exchanged heteropolyacid (where A in formula II is the cation). Examples of A (the cation) in formula II include metal ions such as lithium, sodium, potassium, cesium, magnesium, barium, copper, rubidium, thallium, gold or gallium ions; thus the heteropolyacid salt can be a heteropolyacid metal salt. Other examples of A in formula II include onium groups such as ammonium (NH₄⁺) and organic amine. The cation in certain embodiments of formula II may be selected on the basis that the cation-exchanged heteropolyacid is acidic and insoluble in water. Thus, the catalyst composition in certain embodiments comprises a heteropolyacid salt that is acidic and insoluble in water. Such a heteropolyacid salt can be a cesium-exchanged heteropolyacid (heteropolyacid cesium salt), for example. It would be understood in the art that the cesium ion in such heteropolyacid salts represents the A group in formula II. The A group in certain other embodiments can alternatively be potassium or ammonium. In certain embodiments, j+k≦4 in formula II.

The heteropolyacid or heteropolyacid salt in certain embodiments of the disclosed invention comprises tungsten (W). It would be understood in the art that the tungsten in such heteropolyacids represents peripheral element M in either formula I or II. The heteropolyacid or heteropolyacid salt in certain embodiments comprises phosphorus (P) or silicon (Si). It would be understood in the art that the phosphorus or silicon in such heteropolyacids represents center element X in either formula I or II.

Examples of heteropolyacids that can be used include H₃PW₁₂O₄₀. and H₄SiW₁₂O₄₀, which both follow formula I. Examples of heteropolyacid salts include Cs_{2.5}H_{0.5}PW₁₂O₄₀ and Cs_{2.5}H_{0.5}SiW₁₂O₄₀, which both follow formula II.

The heteropolyacid in certain embodiments can be either anhydrous or hydrated. Hydrated heteropolyacids (i.e., containing crystallization water) include H₃PW₁₂O₄₀.(H₂O)ₓ and H₄SiW₁₂O₄₀.(H₂O)ₓ, for example, where x is equal to or greater than 1. However, in certain embodiments, a hydrated heteropolyacid is dehydrated before using it to prepare the disclosed catalyst compositions.

The catalyst composition in certain embodiments of the disclosed invention can be prepared by supporting the heteropolyacid or heteropolyacid salt component on the metal catalyst component. This can be accomplished, for example, by precipitating a heteropolyacid in the presence of a metal catalyst. In general, such a process can include (i) mixing the metal catalyst in an aqueous heteropolyacid solution and (ii) adding a salt solution that precipitates the heteropolyacid thereby creating a precipitate. The metal catalyst in certain embodiments does not dissolve in the aqueous solution; hence, the salt in step (ii) would be added to a mixture of undissolved metal catalyst and dissolved heteropolyacid. The salt may be added while agitating the mixture, which keeps the metal catalyst in suspension; such would result in a precipitate that more uniformly contains the metal catalyst component and the precipitated heteropolyacid salt component.

In certain embodiments, the aqueous heteropolyacid solution is prepared by first dehydrating the heteropolyacid (if hydrated) using heat (e.g., about 60° C.) and/or vacuum for a suitable amount of time (e.g., about 2 hours), for example. The dehydrated heteropolyacid is then dissolved in water to provide an aqueous solution to which the metal catalyst is then added.

The salt used to precipitate the heteropolyacid (“heteropolyacid-precipitating salt”) in the presence of the metal catalyst can first be dried under high heat (e.g., about 420° C.) and/or a vacuum for a suitable amount of time (e.g., about 2 hours), for example, before preparing a solution of the salt.

The heteropolyacid-precipitating salt in certain embodiments can contain the element or group represented by A in formula II. For example, the salt can be one or more of a carbonate, hydroxide, sulfate, or sulfide of any of the elements or groups represented by A in formula II. Cs₂CO₃ is an example of a carbonate that could be used as the heteropolyacid-precipitating salt. It would be understood that a catalyst composition prepared using this precipitation process contains an insoluble heteropolyacid salt having formula II.

The precipitated catalyst composition in certain embodiments may be dried using heat (e.g. about 100-150° C. or 120° C.) and/or vacuum conditions to remove water for a suitable amount of time (e.g., about 2 hours). This dried product may further be calcined in certain embodiments at a temperature of about 250-350° C. (e.g., about 300° C.) for a suitable amount of time (e.g., about 1 hour).

The ratio of the metal catalyst component to the heteropolyacid salt component in a catalyst composition prepared using a precipitation process can be about 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 4.0, or 5.0 parts of the metal catalyst to about 1 part of the heteropolyacid salt, where each component is measured on a weight basis. The ratio of the metal catalyst to the heteropolyacid salt is about 1:1 in certain embodiments.

A catalyst composition prepared via precipitation may comprise, for example, a metal catalyst containing copper and manganese. Such a metal catalyst may contain CuO and/or MnO₂; a particular example is CuO/MnO₂/Al₂O₃. This and other catalyst compositions prepared via precipitation may comprise a cesium-, potassium-, or ammonium-heteropolyacid salt, for example. Such a heteropolyacid salt may comprise silicon or phosphorus as the center element and tungsten as the peripheral element, for example (e.g., Cs_{2.5}H_{0.5}PW₁₂O₄₀).

Another way, for example, that the heteropolyacid component can be supported on the metal catalyst component of the catalyst composition is through impregnation (e.g., wet impregnation). In general, the metal catalyst component is mixed in a heteropolyacid solution and then the mixture dried down to solids. This drying step may be done under moderate heat (e.g., about 40-100° C.), under a vacuum, and/or under elevated heat (e.g., about 100-150° C.), for example. The dried product may also be calcined in certain embodiments at a temperature of about 250-1000° C. (e.g., about 300° C.) for a suitable amount of time (e.g., about 1 hour).

In certain embodiments of the disclosed invention, the catalyst composition can be prepared by mixing the metal catalyst component with the heteropolyacid or heteropolyacid salt component under dry conditions. In other words, the mixing is performed without the introduction of water or any solution. For example, a dry amount of a metal catalyst and a dry amount of a heteropolyacid or heteropolyacid salt are placed together and ground down to a powder. The resulting powder contains fine particles of the metal catalyst and the heteropolyacid or heteropolyacid salt. This mixing can also be referred to as intimate mixing. Thus, in certain embodiments, the catalyst composition comprises a dry mixture of the metal catalyst and the heteropolyacid or heteropolyacid salt. This mixture can also be referred to as a fine mixture, intimate mixture, or a powder mixture.

In alternative embodiments, the mixing can be performed by mixing together metal catalyst and heteropolyacid components that have each already been rendered into a powder (i.e., metal catalyst powder is mixed with heteropolyacid powder). Still alternatively, one component not previously rendered as a powder may be placed with the other component that has already been rendered as a powder, after which the first component is ground down (e.g., metal catalyst powder and non-powderized heteropolyacid salt may be placed together and subject to grinding/powderization).

Depending on the size of the operation, dry-mixing can be performed using an industrial grinder as known in the art, or on a smaller scale using a mortar and pestle, for example. Dry-mixing can be performed in certain embodiments such that a certain amount of pressure or force is applied by the mixing device to the particles being mixed. For example, the mixing device may apply force of about 100 to 500 pounds (e.g., about 300 pounds) to the components being mixed. The force applied to the components being mixed does not need to be uniformly applied throughout the mixing process.

The mixing process can be carried out for any suitable amount of time. For example, mixing of the metal catalyst and heteropolyacid components may be performed for at least about 1, 2, 3, 4, 5, 6, 7, 8, 9, or 10 minutes. Catalyst compositions prepared by dry-mixing may be used in a hydrodeoxygenation reaction immediately after preparation (e.g. within about 15 or 30 minutes), or may be stored for later use, preferably in an inert atmosphere.

The catalyst composition prepared by dry-mixing the metal catalyst and heteropolyacid components may comprise particles having an average mesh size (U.S. standard scale) of about 140, 130, 120, 110, 100, 90, 80, 70, 60, 50, or 40, for example (or about 140 to 40 mesh). In certain embodiments, the average particle size (e.g., longest length dimension) is about 150, 160, 170, 180, 190, 200, 210, 220, 230, 240 or 250 microns (or about 150-250 microns). Thus, a dry mixture, fine mixture, or intimate mixture can refer to a mix of metal catalyst and heteropolyacid/heteropolyacid salt particles having an average mesh size or particle size as listed above.

The term “dry mixture” is meant to refer to the state of the catalyst composition upon its production. In this sense, the catalyst composition in certain embodiments can be one that was produced by dry-mixing. It would be understood that use of the dry mixture in the disclosed hydrodeoxygenation process exposes it to liquids such as solvents, certain feedstocks and/or certain products.

The heteropolyacid used to prepare a catalyst composition by dry-mixing in certain embodiments may be a heteropolyacid salt. The heteropolyacid salt may be prepared in any manner known in the art. For example, the heteropolyacid salt can be prepared by first dehydrating a heteropolyacid (if hydrated) using heat (e.g., about 60° C.) and/or vacuum for a suitable amount of time (e.g., about 2 hours). The heteropolyacid (dehydrated or not) is then dissolved in water to provide an aqueous heteropolyacid solution. Prior to its use in precipitating the heteropolyacid, the heteropolyacid-precipitating salt can be dried in certain embodiments under high heat (e.g., about 420° C.) and/or a vacuum for a suitable amount of time (e.g., about 2 hours). A solution of the heteropolyacid-precipitating salt is then added to the heteropolyacid solution to produce a heteropolyacid salt precipitate. The heteropolyacid salt in certain embodiments can be dried down under moderate heat (e.g., about 40-100° C.), under a vacuum, and/or under elevated heat (e.g., about 100-150° C.) for a suitable amount of time (e.g., about 2 hours), for example. The dried heteropolyacid salt may also be calcined in certain embodiments at a temperature of about 250-1000° C. (e.g., about 300° C.) for a suitable amount of time (e.g., about 1 hour).

The ratio of the metal catalyst component to the heteropolyacid component in a catalyst composition prepared by a dry-mixing process can be about 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 4.0, or 5.0 parts of the metal catalyst to about 1 part of the heteropolyacid or heteropolyacid salt, where each component is measured on a weight basis. The ratio of the metal catalyst to the heteropolyacid or heteropolyacid salt is about 0.6:1, 1:1, or 2:1 in certain embodiments.

A catalyst composition prepared via dry-mixing may comprise, for example, a heteropolyacid such as H₃PW₁₂O₄₀. or H₄SiW₁₂O₄₀ and a metal catalyst containing (i) CuO and MnO₂, (ii) CuO, BaO and Cr₂O₃, or (iii) CuO, BaO, Cr₂O₃ and MnO₂. Particular examples of such metal catalysts include CuO/MnO₂/Al₂O₃, BaO/CuO/Cr₂O₃/SiO₂ and BaO/CuO/MnO₂/Cr₂O₃.

Alternatively, the catalyst composition prepared via dry-mixing may comprise, for example, a heteropolyacid salt such as Cs_{2.5}H_{0.5}PW₁₂O₄₀ or Cs_{2.5}H_{0.5}SiW₁₂O₄₀ and a metal catalyst containing (i) CuO and MnO₂ or (ii) CuO, BaO, Cr₂O₃ and MnO₂. Particular examples of such metal catalysts include CuO/MnO₂/Al₂O₃ and BaO/CuO/MnO₂/Cr₂O₃.

In certain embodiments, the heteropolyacid or heteropolyacid salt component in a catalyst composition prepared using any of the disclosed processes is not covalently linked to the metal catalyst, whereas in other embodiments they are covalently linked to the metal catalyst component. The covalent linkage can be a result of a calcination step, for example.

The linear alkanes produced by the disclosed invention are suitable for use in producing long-chain diacids by fermentation. For example, the linear alkanes may be fermented, individually or in combination, to linear dicarboxylic acids of 10 (decanedioic acid), 12 (dodecanedioic acid), 14 (tetradecanedioic acid), 16 (hexadecanedioic acid), or 18 (octadecanedioic acid) carbons in length. Methods and microorganisms for fermenting linear alkanes to linear dicarboxylic acids are described, for example, in U.S. Pat. Nos. 5,254,466; 5,620,878; 5,648,247, and U.S. Pat. Appl. Publ. Nos. 2011-0300594, 2005-0181491 and 2004-0146999 (all of which are herein incorporated by reference). Methods for recovering linear dicarboxylic acids from fermentation broth are also known, as disclosed in some of the above references and also in U.S. Pat. No. 6,288,275 and International Pat. Appl. Publ. No. WO2000-020620.

### EXAMPLES

The disclosed invention is further defined in the following Examples. It should be understood that these Examples, while indicating certain preferred aspects of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The following materials were used to prepare the catalyst compositions disclosed in the below examples and which are listed in Table 2.

Cs₂CO₃, H₃PW₁₂O₄₀.(H₂O)ₓ and H₄SiW₁₂O₄₀.(H₂O)ₓ were purchased from Sigma-Aldrich (St. Louis, Mo.).

SiO₂ powder mesh 60 was purchased from EMD (Merck KGaA, Darmstadt, Germany).

CuO/MnO₂/Al₂O₃ (Cat. No. T-4489), BaO/CuO/Cr₂O₃/SiO₂ (Cat. No. G-22/2) and BaO/CuO/MnO₂/Cr₂O₃ (Cat. No. G-99B-0) were received from Süd-Chemie (Louisville, Ky.). The CuO/MnO₂/Al₂O₃ as provided contained 56% CuO, 10% MnO₂ and 34% Al₂O₃ by weight. The BaO/CuO/Cr₂O₃/SiO₂ as provided contained 6% BaO, 47% CuO, 34% Cr₂O₃ and 13% SiO₂ by weight. The BaO/CuO/MnO₂/Cr₂O₃ as provided contained 2% BaO, 47% CuO, 4% MnO₂ and 46% Cr₂O₃. These metal catalysts were prepared by co-precipitation.

CuO/SiO₂ (Cat. No. Cu-0860) was received from BASF. The CuO/SiO₂ as provided contained 30-50% decan-1-ol, 25-40% copper, 10-20% SiO₂, 0-10% CaO, 0-10% CuO, 0-7% palygorskite and 0-1% crystalline silica.

Zeolite (SiO₂/Al₂O₃) (Cat. No. CBV780) was purchased from Zeolyst Intemational (Conshohocken, Pa.). The mole ratio of the SiO₂ to Al₂O₃ in the zeolite was 80:1.

ZrO₂ and ZrO₂WO₃ (Cat. No. XZO 1250) were purchased from MEL Chemicals (Flemington, N.J.). The ZrO₂WO₃ contained 15% WO₃ (on ZrO₂ basis).

### Example 1

### Preparation of Partially Cs-Exchanged Heteropolyacids

This Example describes the general procedure for preparing the partially Cs-exchanged heteropolyacids Cs_{2.5}H_{0.5}PW₁₂O₄₀ and Cs_{2.5}H_{0.5}SiW₁₂O₄₀. These heteropolyacid salts were used to prepare various catalyst compositions comprising heteropolyacid salt and metal catalyst components. The resulting catalyst compositions can be used in hydrodeoxygenation reaction procedures.

The cesium salts of the tungsten heteropolyacids were prepared using an aqueous solution of Cs₂CO₃ and an aqueous solution of either H₃PW₁₂O₄₀ or H₄SiW₁₂O₄₀. The heteropolyacid H₃PW₁₂O₄₀ or H₄SiW₁₂O₄₀ was prepared for use in aqueous solution by first dehydrating it at 60° C. under a vacuum for 2 hours. Cs₂CO₃ was dehydrated at 420° C. for 2 hours under a vacuum prior to its use for preparing an aqueous solution.

Partially Cs-exchanged heteropolyacids were prepared by titrating an aqueous solution of H₃PW₁₂O₄₀ (0.08 mol dm⁻³) or H₄SiW₁₂O₄₀ (0.08 mol dm⁻³) with an aqueous solution of Cs₂CO₃ (0.25 mol/L) at room temperature at a rate of 1 mL/minute. The resulting white colloidal suspension (precipitated heteropolyacid salt) was evaporated to a solid at 50° C. under a vacuum. The solids were then placed in a 120° C. vacuum oven for 2 hours to remove water. The dried solids were optionally calcined in air at 300° C. for 1 hour.

Thus, the partially Cs-exchanged heteropolyacids Cs_{2.5}H_{0.5}PW₁₂O₄₀ and Cs_{2.5}H_{0.5}SiW₁₂O₄₀ were prepared. Example 3 below describes using these heteropolyacid salts directly in a dry mixing process to prepare certain catalyst compositions.

### Example 2

### Preparation of Partially Cs-Exchanged Heteropolyacids Supported on Metal Catalysts or Other Material

This Example describes the general procedure for preparing partially Cs-exchanged heteropolyacids (Cs_{2.5}H_{0.5}PW₁₂O₄₀ and Cs_{2.5}H_{0.5}SiW₁₂O₄₀) supported on SiO₂ or a metal catalyst (CuO/MnO₂/Al₂O₃). The resulting supported heteropolyacid catalyst compositions were used in the hydrodeoxygenation procedure described in Example 5.

Where SiO₂ was used as the support, 15 parts by weight of Cs_{2.5}H_{0.5}PW₁₂O₄₀ or Cs_{2.5}H_{0.5}SiW₁₂O₄₀ were supported on 85 parts by weight of SiO₂. Where a metal catalyst (CuO/MnO₂/Al₂O₃) was used as the support, 1 or 5 parts by weight of CuO/MnO₂/Al₂O₃ were used to support 1 part by weight of Cs_{2.5}H_{0.5}PW₁₂O₄₀ or Cs_{2.5}H_{0.5}SiW₁₂O₄₀.

The procedure for preparing the SiO₂-supported and metal catalyst-supported heteropolyacid salt catalysts was similar to that described in Example 1, as follows.

The heteropolyacid H₃PW₁₂O₄₀ or H₄SiW₁₂O₄₀ was prepared for use in aqueous solution by first dehydrating it at 60° C. under a vacuum for 2 hours. Cs₂CO₃ was dehydrated at 420° C. for 2 hours under a vacuum prior to its use for preparing an aqueous solution.

Partially Cs-exchanged heteropolyacids supported on SiO₂ or CuO/MnO₂/Al₂O₃ were prepared by first suspending the appropriate amount of the support material (depending on the heteropolyacid:support ratio of the final product) in the aqueous solution of H₃PW₁₂O₄₀ (0.08 mol dm⁻³) or H₄SiW₁₂O₄₀ (0.08 mol dm⁻³). This mixture was then titrated with an aqueous solution of Cs₂CO₃ (0.25 mol/L) at room temperature at a rate of 1 mL/minute to precipitate the heteropolyacid. The resulting colloidal suspension was evaporated to a solid at 50° C. under a vacuum. The solids were then placed in a 120° C. vacuum oven for 2 hours to remove water. The dried solids were optionally calcined in air at 300° C. for 1 hour.

Thus, the partially Cs-exchanged heteropolyacids Cs_{2.5}H_{0.5}PW₁₂O₄₀ and Cs_{2.5}H_{0.5}SiW₁₂O₄₀ (heteropolyacid cesium salts) were supported on either SiO₂ or the metal catalyst CuO/MnO₂/Al₂O₃. The process for preparing these catalysts involved mixing the support material in an aqueous heteropolyacid solution before precipitating the heteropolyacid with Cs₂CO₃.

Other metal catalysts such as BaO/CuO/Cr₂O₃/SiO₂, BaO/CuO/MnO₂/Cr₂O₃ and CuO/SiO₂, for example, can be used in the above procedure (instead of CuO/MnO₂/Al₂O₃) to prepare catalysts containing heteropolyacid salts.

### Example 3

### Preparation of Catalysts by Dry-Mixing Metal Catalysts with a Heteropolyacid or Partially Cs-Exchanged Heteropolyacid

This Example describes the general procedure for preparing catalysts by physically mixing a metal catalyst (CuO/MnO₂/Al₂O₃, BaO/CuO/Cr₂O₃/SiO₂, BaO/CuO/MnO₂/Cr₂O₃, CuO/SiO₂) with a heteropolyacid (H₃PW₁₂O₄₀ or H₄SiW₁₂O₄₀), a partially Cs-exchanged heteropolyacid (Cs_{2.5}H_{0.5}PW₁₂O₄₀ or Cs_{2.5}H_{0.5}SiW₁₂O₄₀), or other catalyst component (zeolite, ZrO₂/WO₃, ZrO₂). The resulting catalyst compositions were used in the hydrodeoxygenation procedure described in Example 5.

A dry amount of a metal catalyst (CuO/MnO₂/Al₂O₃, BaO/CuO/Cr₂O₃/SiO₂, BaO/CuO/MnO₂/Cr₂O₃, or CuO/SiO₂) was combined with a dry amount of a heteropolyacid (H₃PW₁₂O₄₀ or H₄SiW₁₂O₄₀), a partially Cs-exchanged heteropolyacid (Cs_{2.5}H_{0.5}PW₁₂O₄₀ or Cs_{2.5}H_{0.5}SiW₁₂O₄₀, prepared in Example 1), or other component (zeolite, ZrO₂/WO₃, or ZrO₂). The selected amount of each component was based on the desired ratio of each component, measured by weight, in the resulting catalyst composition (refer to ratios in Table 2, Example 5). The selected components for preparing each catalyst in Table 2 (except reactions 17-20 and 33-40) were combined in a mortar and mixed with a pestle for about 5 minutes. The resulting catalyst mixture was then immediately used in a hydrodeoxygenation process or stored in an inert gas atmosphere before further use. The yield of each catalyst composition was quantitative. A 300° C. (1 hour) calcination step was performed with certain of the catalyst preparations (refer to Table 2).

Thus, catalyst compositions were prepared by intimately mixing metal catalysts with heteropolyacids or heteropolyacid salts.

### Example 4

### Preparation of Catalysts by Wet Impregnation of Metal Catalysts or Other Materials with Heteropolyacids

This Example describes the general procedure for preparing catalysts prepared through wet impregnation of metal catalysts (e.g., CuO/MnO₂/Al₂O₃) or other support material (e.g., SiO₂) with heteropolyacids (e.g., H₃PW₁₂O₄₀ or H₄SiW₁₂O₄₀). This procedure resulted in the preparation of supported heteropolyacid catalyst compositions.

Twenty to 50 parts of a metal catalyst (CuO/MnO₂/Al₂O₃, BaO/CuO/Cr₂O₃/SiO₂, BaO/CuO/MnO₂/Cr₂O₃, or CuO/SiO₂) or another material were combined with 80 to 50 parts of an aqueous solution of a heteropolyacid (H₃PW₁₂O₄₀ or H₄SiW₁₂O₄₀), depending on the desired ratio of metal catalyst or other material to heteropolyacid. The resulting suspension was evaporated to relative dryness at 40-50° C. This composition was then dried at 80° C. under a vacuum with vigorous mixing to remove the remaining water, after which it was finally dried in a vacuum oven at 120° C. and optionally calcined in air at 300° C. for 1 hour.

Thus, heteropolyacids were supported on either a metal catalyst or other material via a wet impregnation technique.

### Example 5

### Hydrodeoxygenation of Oxygenated Feedstock Using Catalyst Compositions Comprising Metal Catalysts and Heteropolyacids

This Example describes using certain of the catalyst compositions prepared in the above examples in a hydrodeoxygenation reaction process to produce an alkane from oxygenated feedstock. Specifically, n-dodecanol was hydrodeoxygenated to dodecane in reactions catalyzed by various catalyst compositions comprising a metal catalyst and a heteropolyacid or heteropolyacid cesium salt.

Each reaction was performed as follows. Tetradecane (400 mg) was added to 100 mg of n-dodecanol and about 75 mg of a particular catalyst composition (discussed below) in a glass vial equipped with a magnetic stir bar. The vial was capped with a perforated septum to limit vapor transfer rates. Next, the capped vial was placed in a stainless steel (SS316) parallel pressure reactor (8 individual wells). The reactor was then connected to a high pressure gas manifold and purged with nitrogen gas (1000 psi) three times before hydrogen was added. About 700 psi of hydrogen was added and the reactor was heated to 260° C., after which the hydrogen pressure in the reactor was adjusted to about 1000 psi. These conditions were held for 4 hours.

The reactor was then allowed to cool to room temperature and the pressure was released. A 100-μL sample was taken from each vial, diluted with n-propanol containing an internal standard, filtered through a 5-micron disposable filter, and analyzed by GC (and in some cases GC/MS) using an internal standard method for quantitative analysis. Results for each reaction are provided in Table 2.

A total of 44 different reactions were performed using various catalyst compositions prepared using the procedures described in Examples 2 or 3. The components, and the ratios of each component, in each catalyst composition are indicated in Table 2. Specifically, each catalyst composition (except for reactions 33-36) comprised a metal catalyst (CuO/MnO₂/Al₂O₃, BaO/CuO/Cr₂O₃/SiO₂, BaO/CuO/MnO₂/Cr₂O₃, CuO/SiO₂) and another component (H₃PW₁₂O₄₀, H₄SiW₁₂O₄₀, Cs_{2.5}H_{0.5}PW₁₂O₄₀, H₃PO₄, zeolite, ZrO₂/WO₃, ZrO₂). Table 2 further indicates the process (Example 2 or 3) used to prepare the catalyst composition, and whether the catalyst composition was calcined after its preparation. As described above, each reaction listed in Table 2 was performed for 4 hours at 260° C. under 1000 psi of hydrogen gas.

TABLE 2
Hydrodeoxygenation of Dodecanol to Dodecane Using Catalyst Compositions Comprising a Metal Catalyst and a
Heteropolyacid, Heteropolyacid Salt, or Other Coponent

| | Catalyst Composition | | | | | Dodecane | | Didodecylether | | Dodecanol |
|---|---|---|---|---|---|---|---|---|---|---|
| Rxn | Metal Catalyst | Heteropolyacid or | | Prep. | Calc. | Selectivity | Yield | Selectivity | Yield | Conversion |
| No. | Component | Other Component^{a} | Ratio^{b} | Method | (° C.)^{c} | (%)^{e} | (%) | (%)^{e} | (%) | (%)^{f} |
| 1 | CuO/MnO₂/Al₂O₃ | H₃PW₁₂O₄₀ | 2:1 | Ex. 3 | n/a | 92.3 | 91.4 | 1.1 | 1.1 | 99.0 |
| 2 | BaO/CuO/Cr₂O₃/SiO₂ | H₃PW₁₂O₄₀ | 2:1 | Ex. 3 | n/a | 82.7 | 82.4 | 0.0 | 0.0 | 99.7 |
| 3 | BaO/CuO/MnO₂/Cr₂O₃ | H₃PW₁₂O₄₀ | 2:1 | Ex. 3 | n/a | 68.7 | 68.7 | 0.0 | 0.0 | 100.0 |
| 4 | CuO/SiO₂ | H₃PW₁₂O₄₀ | 2:1 | Ex. 3 | n/a | 39.6 | 8.4 | 8.8 | 1.9 | 21.4 |
| 5 | CuO/MnO₂/Al₂O₃ | H₄SiW₁₂O₄₀ | 2:1 | Ex. 3 | n/a | 80.9 | 76.8 | 3.7 | 3.5 | 95.0 |
| 6 | BaO/CuO/Cr₂O₃/SiO₂ | H₄SiW₁₂O₄₀ | 2:1 | Ex. 3 | n/a | 66.9 | 59.6 | 6.5 | 5.8 | 89.0 |
| 7 | BaO/CuO/MnO₂/Cr₂O₃ | H₄SiW₁₂O₄₀ | 2:1 | Ex. 3 | n/a | 90.8 | 90.5 | 0.0 | 0.0 | 99.7 |
| 8 | CuO/SiO₂ | H₄SiW₁₂O₄₀ | 2:1 | Ex. 3 | n/a | 27.1 | 3.6 | 6.1 | 0.8 | 13.2 |
| 9 | CuO/MnO₂/Al₂O₃ | Cs_{2.5}H_{0.5}PW₁₂O₄₀^{d} | 1:1 | Ex. 3 | 300 | 93.2 | 93.2 | 0.0 | 0.0 | 100.0 |
| 10 | BaO/CuO/Cr₂O₃/SiO₂ | Cs_{2.5}H_{0.5}PW₁₂O₄₀^{d} | 1:1 | Ex. 3 | 300 | 20.1 | 3.7 | 17.9 | 3.3 | 18.5 |
| 11 | BaO/CuO/MnO₂/Cr₂O₃ | Cs_{2.5}H_{0.5}PW₁₂O₄₀^{d} | 1:1 | Ex. 3 | 300 | 86.6 | 84.6 | 8.3 | 8.1 | 97.7 |
| 12 | CuO/SiO₂ | Cs_{2.5}H_{0.5}PW₁₂O₄₀^{d} | 1:1 | Ex. 3 | 300 | 20.2 | 3.8 | 42.3 | 3.5 | 19.0 |
| 13 | CuO/MnO₂/Al₂O₃ | Cs_{2.5}H_{0.5}PW₁₂O₄₀ | 1:1 | Ex. 3 | n/a | 94.8 | 94.8 | 0.0 | 0.0 | 100.0 |
| 14 | BaO/CuO/Cr₂O₃/SiO₂ | Cs_{2.5}H_{0.5}PW₁₂O₄₀ | 1:1 | Ex. 3 | n/a | 33.8 | 13.0 | 30.4 | 11.7 | 38.5 |
| 15 | BaO/CuO/MnO₂/Cr₂O₃ | Cs_{2.5}H_{0.5}PW₁₂O₄₀ | 1:1 | Ex. 3 | n/a | 88.3 | 88.3 | 0.0 | 0.0 | 100.0 |
| 16 | CuO/SiO₂ | Cs_{2.5}H_{0.5}PW₁₂O₄₀ | 1:1 | Ex. 3 | n/a | 23.7 | 3.7 | 45.5 | 7.1 | 15.7 |
| 17 | CuO/MnO₂/Al₂O₃ | H₃PO₄ | 5:2 | n/a | n/a | 0.0 | 0.0 | 0.0 | 0.0 | 10.2 |
| 18 | BaO/CuO/Cr₂O₃/SiO₂ | H₃PO₄ | 5:2 | n/a | n/a | 0.0 | 0.0 | 8.8 | 1.5 | 16.7 |
| 19 | BaO/CuO/MnO₂/Cr₂O₃ | H₃PO₄ | 5:2 | n/a | n/a | 0.0 | 0.0 | 0.0 | 0.0 | 9.3 |
| 20 | CuO/SiO₂ | H₃PO₄ | 5:2 | n/a | n/a | 4.3 | 1.4 | 22.3 | 7.4 | 33.2 |
| 21 | CuO/MnO₂/Al₂O₃ | zeolite | 1:1 | Ex. 3 | n/a | 94.4 | 94.4 | 0.0 | 0.0 | 100.0 |
| 22 | BaO/CuO/Cr₂O₃/SiO₂ | zeolite | 1:1 | Ex. 3 | n/a | 0.0 | 0.0 | 13.8 | 3.0 | 21.7 |
| 23 | BaO/CuO/MnO₂/Cr₂O₃ | zeolite | 1:1 | Ex. 3 | n/a | 29.1 | 9.5 | 17.2 | 5.6 | 32.7 |
| 24 | CuO/SiO₂ | zeolite | 1:1 | Ex. 3 | n/a | 0.0 | 0.0 | 21.1 | 3.8 | 18.2 |
| 25 | CuO/MnO₂/Al₂O₃ | ZrO₂/WO₃ | 1:1 | Ex. 3 | n/a | 0.0 | 0.0 | 14.6 | 2.5 | 16.9 |
| 26 | BaO/CuO/Cr₂O₃/SiO₂ | ZrO₂/WO₃ | 1:1 | Ex. 3 | n/a | 0.0 | 0.0 | 8.8 | 1.5 | 16.7 |
| 27 | BaO/CuO/MnO₂/Cr₂O₃ | ZrO₂/WO₃ | 1:1 | Ex. 3 | n/a | 0.0 | 0.0 | 23.0 | 3.7 | 16.0 |
| 28 | CuO/SiO₂ | ZrO₂/WO₃ | 1:1 | Ex. 3 | n/a | 0.0 | 0.0 | 20.8 | 3.2 | 15.4 |
| 29 | CuO/MnO₂/Al₂O₃ | ZrO₂ | 1:1 | Ex. 3 | n/a | 0.0 | 0.0 | 0.0 | 0.0 | 13.5 |
| 30 | BaO/CuO/Cr₂O₃/SiO₂ | ZrO₂ | 1:1 | Ex. 3 | n/a | 0.0 | 0.0 | 0.0 | 0.0 | 13 |
| 31 | BaO/CuO/MnO₂/Cr₂O₃ | ZrO₂ | 1:1 | Ex. 3 | n/a | 0.0 | 0.0 | 0.0 | 0.0 | 13.2 |
| 32 | CuO/SiO₂ | ZrO₂ | 1:1 | Ex. 3 | n/a | 0.0 | 0.0 | 0.0 | 0.0 | 13.4 |
| 33 | CuO/MnO₂/Al₂O₃ | none | n/a | n/a | n/a | 0.0 | 0.0 | 0.0 | 0.0 | 13.6 |
| 34 | BaO/CuO/Cr₂O₃/SiO₂ | none | n/a | n/a | n/a | 0.0 | 0.0 | 0.0 | 0.0 | 13.8 |
| 35 | BaO/CuO/MnO₂/Cr₂O₃ | none | n/a | n/a | n/a | 0.0 | 0.0 | 0.0 | 0.0 | 13.2 |
| 36 | CuO/SiO₂ | none | n/a | n/a | n/a | 0.0 | 0.0 | 0.0 | 0.0 | 12.4 |
| 37 | CuO/MnO₂/Al₂O₃ | Cs_{2.5}H_{0.5}PW₁₂O₄₀ | 1:1 | Ex. 2 | n/a | 88.5 | 88.5 | 0.6 | 0.6 | 100 |
| 38 | CuO/MnO₂/Al₂O₃ | Cs_{2.5}H_{0.5}PW₁₂O₄₀ | 1:1 | Ex. 2 | 300 | 88.7 | 88.7 | 0.1 | 0.1 | 100 |
| 39 | CuO/MnO₂/Al₂O₃ | Cs_{2.5}H_{0.5}PW₁₂O₄₀ | 5:1 | Ex. 2 | n/a | 0.0 | 0.0 | 7.5 | 1.3 | 16.8 |
| 40 | CuO/MnO₂/Al₂O₃ | Cs_{2.5}H_{0.5}PW₁₂O₄₀ | 5:1 | Ex. 2 | 300 | 5.2 | 0.9 | 7.5 | 1.3 | 16.8 |
| 41 | CuO/MnO₂/Al₂O₃ | Cs_{2.5}H_{0.5}PW₁₂O₄₀ | 1:1 | Ex. 3 | n/a | 90.5 | 90.5 | 0.0 | 0.0 | 100 |
| 42 | CuO/MnO₂/Al₂O₃ | Cs_{2.5}H_{0.5}PW₁₂O₄₀^{d} | 1:1 | Ex. 3 | 300 | 82.3 | 82.3 | 0.0 | 0.0 | 100 |
| 43 | CuO/MnO₂/Al₂O₃ | Cs_{2.5}H_{0.5}PW₁₂O₄₀ | 3:5 | Ex. 3 | n/a | 85.9 | 85.9 | 0.0 | 0.0 | 100 |
| 44 | CuO/MnO₂/Al₂O₃ | Cs_{2.5}H_{0.5}PW₁₂O₄₀^{d} | 3:5 | Ex. 3 | 300 | 89.8 | 89.8 | 0.0 | 0.0 | 100 |

^{a}The listed heteropolyacids/salts are H₃PW₁₂O₄₀, H₄SiW₁₂O₄₀, and Cs_{2.5}H_{0.5}PW₁₂O₄₀. The other components listed are H₃PO₄, zeolite, ZrO₂/WO₃ and ZrO₂.
^{b}Ratio of metal catalyst to the heteropolyacid, heteropolyacid salt, or other component.
^{c}Certain catalyst compositions were calcined for 1 hour at 300° C. (see Examples 2 and 3). This calcination step was in addition to the calcination step optionally performed when preparing the Cs-exchanged heteropblyaoid component in certain catalyst cdmpositions (see Footnote d).
^{d}Certain Cs-exchanged heteropolyacids were calcined for 1 hour at 300° C. (see Example 1) before their use in preparing the catalyst compositions containing a metal catalyst.
^{e}Percent moles of dodecanol that converted into dodecane or didodecylether.
^{f}Percent moles of dodecanol that converted to products.

The results listed in Table 2 indicate that catalyst compositions comprising metal catalysts and heteropolyacids or heteropolyacid salts perform well in catalyzing hydrodeoxygenation reactions at low temperature. For several reactions, molar yields of dodecane produced as a result of the complete hydrodeoxygenation of dodecanol were greater than 80%, with some molar yields being greater than 90%. It is apparent that those reactions in Table 2 yielding greater than 90% dodecane also yielded less than 10% by-products such as substituted products (e.g., didodecylether) and products having a carbon chain length shorter than the carbon chain length of the dodecanol substrate.

Table 2 indicates that either a non-modified heteropolyacid (H₃PW₁₂O₄₀ or H₃SiW₁₂O₄₀) or a heteropolyacid salt (Cs_{2.5}H_{0.5}PW₁₂O₄₀) was required for the metal catalyst component of certain catalyst compositions to carry out hydrodeoxygenation. Reactions 33-35, which only used the metal catalysts CuO/MnO₂/Al₂O₃, BaO/CuO/Cr₂O₃/SiO₂ and BaO/CuO/MnO₂/Cr₂O₃, respectively (i.e., no heteropolyacid component), did not produce dodecane from dodecanol. However, the inclusion of a heteropolyacid or heteropolyacid salt with these particular metal catalysts provided catalyst compositions that were effective at hydrodeoxygenating dodecanol (Table 2; reactions 1-3, 5-7, 9, 11, 13, 15, 37, 38, 41-44).

The importance of the heteropolyacid component was further indicated by that when other components (non-heteropolyacid) were included with the metal catalyst component, the resulting catalyst compositions for the most part were not active. For example, catalyst compositions comprising a metal catalyst and H₃PO₄, ZrO₂/WO₃ or ZrO₂ did not have significant levels of hydrodeoxygenation activity (Table 2, reactions 17-20, 25-32). Also, while a catalyst composition comprising zeolite combined with CuO/MnO₂/Al₂O₃ had high hydrodeoxygenation activity (Table 2, reaction 21), inclusion of the other tested metal catalysts with zeolite yielded catalyst compositions having little or no activity.

Table 2 further indicates that metal catalysts can be included with heteropolyacids in different ways and be effective at catalyzing hydrodeoxygenation reactions. For example, the catalysts prepared in Example 2 were prepared by precipitating Cs-exchanged heteropolyacids in the presence of CuO/MnO₂/Al₂O₃. Such catalyst compositions in which the CuO/MnO₂/Al₂O₃ component and the Cs-exchanged heteropolyacid component were at a 1:1 ratio had high hydrodeoxygenation activity (Table 2, reactions 37 and 38). Another way that the disclosed catalyst compositions were prepared involved physical mixing, in the dry state, of metal catalysts with heteropolyacids or heteropolyacid salts through the milling action of a mortar and pestle (Example 3). Examples of such catalysts were shown in reactions 1-3, 5-7, 9, 11, 13, 15 and 41-44 (Table 2) to have hydrodeoxygenation activity. It appears that directly supporting the heteropolyacid component on the metal catalyst component might not be necessary for activity, since physically mixed catalyst compositions that were not calcined (reactions 1-3, 5-7, 13, 15) still catalyzed dodecane production.

Overall, these results demonstrate that a hydrodeoxygenation process employing a catalyst composition comprising a metal catalyst and a heteropolyacid or heteropolyacid salt can be used under low temperature conditions to produce a linear alkane from a C₁₀₋₁₈ oxygenate. The hydrodeoxygenation process with these catalyst compositions mostly yielded the completely deoxygenated, full-length product with a small amount of by-product production.

What is claimed is:

1. A hydrodeoxygenation process for producing a linear alkane from a feedstock comprising a saturated or unsaturated C₁₀₋₁₈ oxygenate comprising a moiety selected from the group consisting of an ester group, carboxylic acid group, carbonyl group, and alcohol group, wherein the process comprises:
a) contacting said feedstock with a catalyst composition at a temperature between about 240° C. to about 280° C. and a hydrogen gas pressure of at least about 300 psi, wherein said catalyst composition comprises (i) a metal catalyst and (ii) a heteropolyacid or heteropolyacid salt, wherein the C₁₀₋₁₈ oxygenate is hydrodeoxygenated to a linear alkane, and wherein the linear alkane has the same carbon chain length as the C₁₀₋₁₈ oxygenate; and
b) optionally, recovering the linear alkane produced in step (a).

2. The hydrodeoxygenation process of claim 1, wherein said C₁₀₋₁₈ oxygenate is a fatty acid or a triglyceride.

3. The hydrodeoxygenation process of claim 1, wherein said feedstock comprises a plant oil or a fatty acid distillate thereof.

4. The hydrodeoxygenation process of claim 3, wherein said feedstock comprises:
(i) a plant oil selected from the group consisting of soybean oil, palm oil and palm kernel oil; or
(ii) a palm fatty acid distillate.

5. The hydrodeoxygenation process of claim 1, wherein said temperature is about 260° C.

6. The hydrodeoxygenation process of claim 1, wherein said pressure is at least about 1000 psi.

7. The hydrodeoxygenation process of claim 1, wherein the metal catalyst comprises copper.

8. The hydrodeoxygenation process of claim 7, wherein the metal catalyst further comprises at least one additional metal selected from the group consisting of manganese, chromium and barium.

9. The hydrodeoxygenation process of claim 1, wherein the heteropolyacid or heteropolyacid salt comprises tungsten.

10. The hydrodeoxygenation process of claim 9, wherein the heteropolyacid or heteropolyacid salt further comprises phosphorus or silicon.

11. The hydrodeoxygenation process of claim 1, wherein the catalyst composition comprises the heteropolyacid salt, and wherein the heteropolyacid salt is acidic and insoluble in water.

12. The hydrodeoxygenation process of claim 11, wherein the heteropolyacid salt is a cesium-exchanged heteropolyacid.

13. The hydrodeoxygenation process of claim 1, wherein the catalyst composition comprises a dry mixture of the metal catalyst and the heteropolyacid or heteropolyacid salt.

14. The hydrodeoxygenation process of claim 1, wherein the molar yield is less than 10% for a reaction product having a carbon chain length that is one or more carbon atoms shorter than the carbon chain length of the C₁₀₋₁₈ oxygenate.
